(19) United States
(12) Patent Application Publication
(10) Pub. No.: US 2008/0142020 A1
(43) Pub. Date: Jun. 19, 2008

(54) Device for protectively covering female genital area

(76) Inventor: Wendy D. Chapin, Fort Bragg, CA (US)

Correspondence Address: EDWIN TARVER, 16830 Ventura Blvd., SUITE 360, Encino, CA, 91436, US

(21) Appl. No.: 11/642,612

(22) Filed: Dec. 19, 2006

(51) Int. Cl. A61F 6/06

(52) U.S. Cl. 128/830

(57) ABSTRACT

A device for protecting female genital area while performing grooming activities such as shaving, waxing and dying of pubic hair. The device comprises a substantially oval-shaped pliable sheet body and an elongated tab extending from a narrow end of the sheet body. The sheet body is symmetric about a longitudinal axis. The dimension of the sheet body along the longitudinal axis is larger than its maximum transverse dimension. The oval shape and size of the sheet body is adapted to cover the female genital area, specifically, the clitoris and the vaginal opening, by securely contour fitting between the labia majoras with the elongated tab pointing downwards. The elongated tab facilitates removal of the device.

### CROSS-REFERENCE TO RELATED APPLICATION

### None

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention relates in general to devices used in contact with the female pubic area, and more particularly to device for covering the female pubic area in order to protect sensitive private parts during grooming activities such as shaving, using depilatories, waxing and dying pubic hair.

Several devices for use with the female private parts have been developed. These devices comprise, for example, pads for collecting body fluids such as menstrual blood, contraceptive vaginal covers, sanitary articles for cleaning pubic area, and the like. A variety of structures and materials were employed for making such devices. U.S. Pat. No. 6,125,850 to Sokal discloses a vaginal device that provides physical and chemical barriers to contraception and protection against sexually transmitted diseases. The device comprises a towelette made of a material having properties of absorbency, penetration, and retention to carry preventive or therapeutic formulations. The towelette may have any desired shape such as square, rectangular, oval or circular, with either smooth or scalloped edges. The towelette is inserted into the vagina before intercourse. A string is attached to towelette so that it can be easily removed after intercourse.

U.S. Pat. No. 5,413,117 to Wills discloses a prophylactic device for humans that allows penetration of body cavities during sexual activity and reduces communication of body fluids between a user and a partner. The device comprises a fluid impervious shield member for draping over the pubic region and a slotted elastic member in a central opening of the shield. The shield member has a fluid absorbing element on one side for capturing body fluids. The slotted elastic member is constructed of a fluid impervious material and has a small slot that allows penetration of the body cavity. The elastic material of the slotted elastic member is sufficiently rigid and flexible to provide squeegee or wiping action on the item being used for penetration to prevent fluids from being withdrawn from the body cavity.

U.S. Pub. No. 20050027277 to Mizutani discloses an absorbent article used in contact with the female pubic region. The article comprises a water permeable sheet facing a body side of a wearer, a water impermeable sheet, an absorbent body between the water permeable water impermeable sheets and a body fluid inflow hole for collecting the body fluid. The opening of the hole is formed to have the size sufficient to be fitted to cover a pair of labia majoras from the outside.

The purpose of the present invention along with its associated design and structure differs from the above devices. Female grooming such as shaving and waxing of the pubic region can be difficult if not dangerous when handling razors and chemicals. Great care has to be taken to protect sensitive genital areas of the pubic region, especially while personally performing such grooming activities. Therefore, it is an object of the present invention to provide a device to protect sensitive genital areas during shaving, waxing and other grooming activities associated with the pubic region.

A further object is to provide a device that provides protection in the form of a contour fitting mask over the female genital area.

Finally, it is an object of the present invention to provide a device that securely fits over the labia majoras. These and other objects of the present invention will become better understood with reference to the appended Summary, Description, and Claims.

### SUMMARY

The present invention is a device for protecting female genital area from chemicals or nicking from razors or electric shavers while performing grooming activities such as shaving, using depilatories, waxing and dying of the pubic hair. The device is designed to cover the genital area, especially the clitoris and the vaginal opening, and uses the contours of the labia majoras to achieve a secure fit. The device comprises a substantially oval shaped sheet body and an elongated tab at one end of the sheet body. An oval shape generally comprises a narrow end and a broad end. The tab extends from the narrow end of the sheet body. The shape and size of the sheet body is configured to facilitate the device to be secured between the labia majoras with the tab pointing down, during grooming activities associated with the pubic region. The sheet body can be trimmed by a user for an accurate fit. The device can also be used to cover the female genital area when tanning in a salon or on a nude beach.

### BRIEF DESCRIPTION OF THE FIGURES

FIG. 1 is a plan view of the device of the present invention.

FIG. 2 is a plan view showing the longitudinal and transverse axes of the device of the present invention.

FIG. 3 is a perspective view of the device of the present invention.

FIG. 4 is a plan view of an alternate embodiment of the device of the present invention that has a different tab design.

FIG. 5 is a perspective view of the device with a folding tab design

FIG. 6 is a perspective view of the device with a fin type tab

FIG. 7 is a perspective view of the device with a string based removal means

### FIGURES—REFERENCE NUMERALS

10 . . . Device of the Present Invention
11 . . . Sheet Body
12 . . . Elongated Tab
13 . . . First End
14 . . . Second End
15 . . . Longitudinal Axis
16 . . . Transverse Axis
17 . . . Alternate Embodiment of the Device
18 . . . Triangular Section
19 . . . Articulating Member
20 . . . Fin-Type Member
21 . . . String

### DETAILED DESCRIPTION

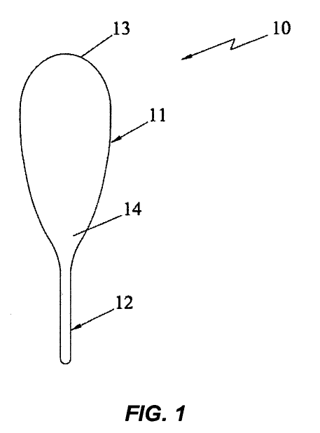

Referring to the drawings, a preferred embodiment of a device for covering female genital area is illustrated and generally indicated as 10 in FIGS. 1 through 3. The device 10 is used during grooming activities associated with the pubic region such as shaving, using depilatories, waxing or dying pubic hair.

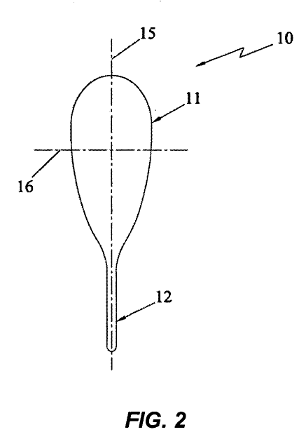

Referring to FIGS. 1 and 2, the device 10 is pliable and comprises a substantially oval shaped sheet body 11 and an elongated tab 12 extending from one of the ends of the sheet body. The oval shaped sheet body 11 has a first end 13 and a second end 14 and is characterized by its symmetry only along its longitudinal axis 15. The second end 14 is narrower than the first end 13. The longitudinal axis 15 in the present case is defined as an axis passing through the centers of the first and second ends and dividing the sheet body 11 into two equal halves. The sheet body 11 is asymmetric about a transverse axis 16 that is defined as an axis perpendicular to the longitudinal axis 15 and dividing the sheet body 11 into two equal halves. The elongated tab 12 extends from the second end 14.

The edges of the sheet body 11 substantially match the contours between the labia majoras. The shape and size of the sheet body 11 is adapted to make it securely fit between the labia majoras for fully cover female genital area, especially the clitoris and the vaginal opening. The device 10 thus protects the clitoris and the vaginal opening from chemicals, razors, electric shavers or the like. The elongated tab 12 facilitates removal of the device.

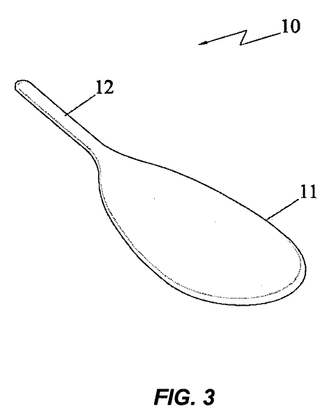

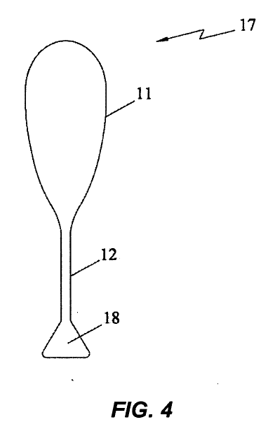

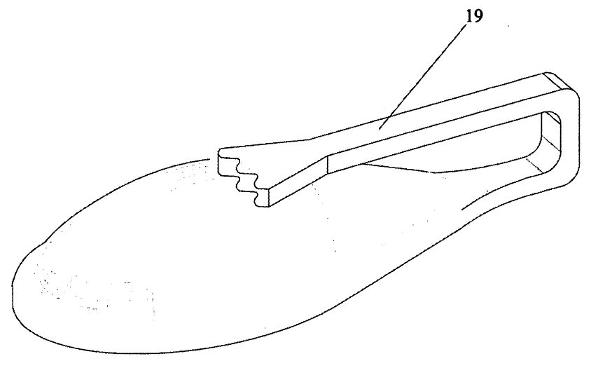

In an alternate embodiment 17 as shown in FIG. 3, the free end of the tab comprises a substantially triangular section 18. The triangular section 18 provides more surface area to facilitate convenient finger placement for removing the device 17.

The device of the present invention is disposable. The sheet body 11 can be trimmed around its edges to achieve an accurate fit. The device is made from hypo-allergenic and safe for skin contact materials.

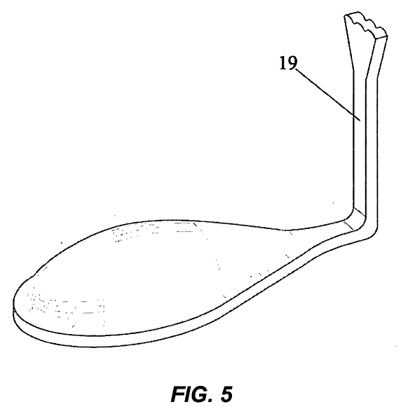

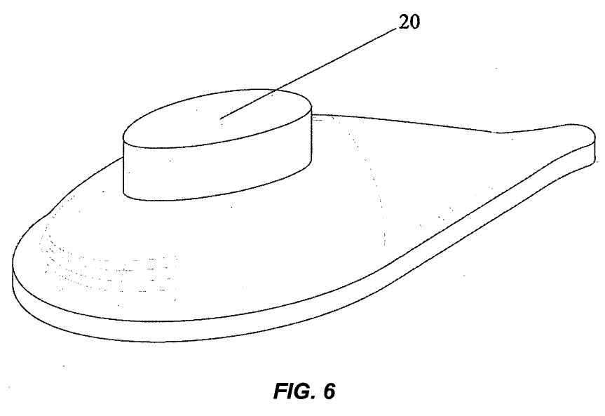

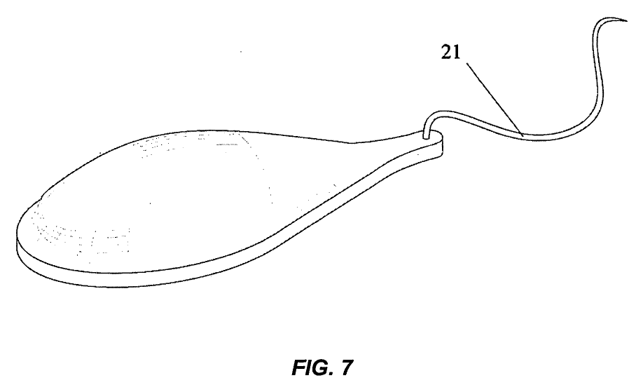

Referring to FIG. 5, in one preferred embodiment, the device can be convex/concave in profile to allow extra room for female genitals. In addition, the device can comprise of a removal means including an articulating member 19 attached to the edge of the device. Referring to FIG. 6, the removal means can include a fin-type member 20 attached to the external face of the device, or a string 21 attached to the device as shown in FIG. 7. In another preferred embodiment, there is no removal means attached to the device.

In another preferred embodiment, the device may comprise of or be coated with an antibacterial and antimicrobial agent. In addition, the device is intended to be made with disposable materials, and may be embossed with logo, run number and or origin of manufacture.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. § 112, paragraph 6. In particular, the use of “step of” in the claims herein is not intended to invoke the provisions of 35 U.S.C. § 112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

1. A device for protecting sensitive areas of the female pubic region during grooming activities including shaving, waxing and dying of pubic hair, comprising:
a. a substantially oval-shaped pliable sheet body, the oval-shaped sheet body being symmetric about a longitudinal axis and asymmetric about a transverse axis perpendicular to the longitudinal axis, each of the axes dividing the sheet body into two halves of equal areas;
b. the dimension of the sheet body along the longitudinal axis being larger than its maximum transverse dimension, the sheet body having a first end and a second end narrower than and opposite the first end along the longitudinal axis, the oval shape and size of the sheet body being adapted to cover the female genital area, specifically, the clitoris and the vaginal opening, by securely contour fitting between the labia majoras with the second end pointing downwards; and
c. an elongated tab extending from the second end of the sheet body that folds back over the sheet body, facilitating removal of the device.

2. The device of claim 1, wherein the sheet body can be trimmed for an accurate fit.

3. The device of claim 1, wherein the device comprises synthetic disposable materials.

4. The device of claim 1, wherein the device comprises natural disposable materials.

5. The device of claim 1, wherein the device is made from hypo-allergenic and safe for skin contact materials.

6. (canceled)

7. A device for protecting sensitive areas of the female pubic region while performing grooming activities such as shaving, waxing and dying of pubic hair, comprising:
a. a drop-shaped pliable sheet body, the sheet body being symmetric about a longitudinal axis and asymmetric about a transverse axis perpendicular to the longitudinal axis, each of the axes dividing the sheet body into two halves of equal areas;
b. the dimension of the sheet body along the longitudinal axis being larger than its maximum transverse dimension, the sheet body having a curved end and a pointed end opposite the curved end along the longitudinal axis, the drop shape and size of the sheet body being adapted to cover the female genital area, specifically, the clitoris and the vaginal opening, by securely contour fitting between the labia majoras with the pointed end facing down; and
c. an elongated tab extending from the pointed end of the sheet body that folds back over the sheet body, facilitating the removal of the device.
